(19) United States
(12) Patent Application Publication
(10) Pub. No.: US 2024/0277491 A1
(43) Pub. Date: Aug. 22, 2024

(54) PROSTHETIC HAND SYSTEM WITH AN INTEGRATED IMAGE RECOGNITION SUBSYSTEM

(71) Applicant: Purdue Research Foundation, West Lafayette, IN (US)

(72) Inventors: Soumya Bandyopadhyay, West Lafayette, IN (US); Justin A. Weibel, West Lafayette, IN (US)

(73) Assignee: Purdue Research Foundation, West Lafayette, IN (US)

(21) Appl. No.: 18/444,401

(22) Filed: Feb. 16, 2024

Related U.S. Application Data
(60) Provisional application No. 63/446,814, filed on Feb. 18, 2023.

(51) Int. Cl. A61F 2/58; A61F 2/70; G06T 1/00; G06T 7/00

(52) U.S. Cl. CPC A61F 2/586; G06T 1/0007; G06T 7/0002; A61F 2002/704

(57) ABSTRACT

A prosthetic hand assembly includes a hand structure including an actuatable wrist, a palm, and a plurality of actuatable fingers coupled with a plurality of actuators each configured to selectively direct movement of the actuatable wrist, a camera disposed on one side of the hand structure, the camera is selectively operable to generate a plurality of successive images of an object positioned adjacent the hand structure, a processor coupled with the plurality of actuators, the processor is communicatively coupled with the camera and configured to receive the plurality of successive images of the object, wherein the processor is configured to determine one or more characteristic of the object from the plurality of successive images of the object, the processor is configured to selectively drive the plurality of actuators to affect the actuatable wrist and plurality of actuatable fingers based on the one or more characteristics.

### CROSS-REFERENCE TO RELATED APPLICATIONS

The present non-provisional patent application is related to and claims the priority benefit of U.S. Provisional patent application Ser. 63/446,814, filed Feb. 18, 2023, the contents of which are hereby incorporated by reference in its entirety into the present disclosure.

### TECHNICAL FIELD

The present disclosure generally relates to robotic prosthesis and in particular to a prosthetic hand with an image recognition system integrated therein.

### STATEMENT REGARDING GOVERNMENT FUNDING

None.

### BACKGROUND

This section introduces aspects that may help facilitate a better understanding of the disclosure. Accordingly, these statements are to be read in this light and are not to be understood as admissions about what is or is not prior art.

Recent Centers for Disease Control and Prevention (CDC) reports suggest that 17% of the children in US are diagnosed with developmental disabilities. Out of 10,000 children in the age group of 4-17, 4-5 are born with upper-limb disabilities. While complete eradication of in-born disabilities is challenging, enabling a child to adjust to any existing disability in the early years of life can significantly enhance the quality of life during adulthood.

Human hand gestures and hand taxonomy have been studied in great detail in the prior art which take note that human fingers comprise bones and tendons. They are actuated by the muscles in the palm and fore-arm through the long elastic tendons linked to the finger bones. According to one researcher, a theoretical model of the human hand was published in the prior art, showing that the fingers, palm, and the wrist have maximum 27 degrees of freedom.

A significant portion of the past work on robotic hand movements rely on studies concerning dexterity. With the anatomy of the human hand and the target gestures for accomplishing specific tasks as a backbone, researchers have started with a mechanical model of robotic hand, representing the phalanges by linkages and the human finger joints by their representative mechanical analogues. The mechanical model has been subsequently employed to devise different actuation techniques for performing different hand gestures. Actuators in robotic hands have also seen variants; while one study shows how a cable-driven actuator can manipulate the robotic hand in almost all the hand gestures highlighted in the taxonomy, other studies employ pneumatic actuators to accomplish the same goals. The use of various actuation mechanisms has consequently resulted in designs ranging between various levels of complexities. On one hand, engineers have designed 20 degrees of freedom (DOF) hands like the Shadow hand that uses pneumatic actuators to perform dexterous anthropomorphic grasping. On the other end, researchers have also designed novel under-actuated (7 DOF) robotic hands using compliant and flexible materials to achieve some level of dexterity.

With this background, to date robotic prosthesis adapted to help a human subject without use of their hand have suffered from various shortcomings. A majority of the previously existing prosthetic hands either have demonstrated open-loop controls and have been targeted towards being implemented on adult human beings. The majority of prosthetic hands come with integrated sensor modalities and incorporate neural feedback to drive the actuators in the system. These systems, however, have an inherent latency making them less than optimal for their intended operation.

Therefore, there is an unmet need for a novel system and method for a prosthetic hand that can eliminate inherent latencies involved in reacting to stimuli.

### SUMMARY

A prosthetic hand assembly is disclosed. The assembly includes a hand structure including an actuatable wrist, a palm, and a plurality of actuatable fingers, wherein the actuatable wrist and at least one of the plurality of actuatable fingers is coupled to corresponding actuators configured to selectively direct movement of the actuatable wrist and at least one of the plurality of actuatable fingers. The prosthetic hand assembly also includes a camera disposed on one side of the hand structure, wherein the camera is selectively operable to generate a plurality of images of an object positioned adjacent the hand structure. The prosthetic hand assembly also includes a processor coupled with the actuators, wherein the processor is communicatively coupled with the camera and configured to receive the plurality of images of the object, wherein the processor is configured to determine one or more characteristic of the object from the plurality of images of the object, and wherein the processor is configured to selectively drive one or more of the actuators to affect movement of the actuatable wrist and at least one of the plurality of actuatable fingers based on the one or more characteristics of the object.

A method of operating a prosthetic hand assembly is also disclosed. The method includes providing a hand structure including an actuatable wrist, a palm, and a plurality of actuatable fingers, wherein the actuatable wrist and at least one of the plurality of actuatable fingers is coupled to corresponding actuators configured to selectively direct movement of the actuatable wrist and at least one of the plurality of actuatable fingers. The method further includes providing a plurality of images of an object adjacent a camera disposed on one side of the hand structure, positioned adjacent the hand structure. Additionally, the method includes communicating the plurality of images to a processor coupled to the actuators, wherein the processor is configured to receive the plurality of images of the object, wherein the processor is configured to determine one or more characteristic of the object from the plurality of images of the object, and wherein the processor is configured to selectively drive one or more of the actuators to affect movement of the actuatable wrist and at least one of the plurality of actuatable fingers based on the one or more characteristics of the object.

### BRIEF DESCRIPTION OF FIGURES

FIG. 1 provides two schematics of a human hand having 27 DOF, translated to the targeted prosthetic hand system of the present disclosure having 20 DOF.

FIG. 2a is a 2D schematic of an actuation mechanism, showing the passage of cables tied to servos, according to the present disclosure.

FIG. 2b is 3D view of an index finger with various features shown in the panel, according to the present disclosure.

FIG. 2c is 3D view of a Thumb with various features, according to the present disclosure.

FIG. 3a is a 3D view of a palm and integrated fingers with a magnified view of the holes in chassis to enable passage of actuation cable, according to the present disclosure.

FIG. 3b is a photograph of an initially assembled prototype with the embedded camera including a palm and five fingers, according to the present disclosure.

FIG. 4a is a 3D view of the integrated wrist, palm and five fingers with various labelled features.

FIG. 4b is a photograph of an initially assembled modular wrist prototype.

FIG. 5a is a pie-chart showing the breakdown of power in Watts, required by electrical and electronic hardware of the present disclosure.

FIG. 5b is a photograph of a battery used in the present disclosure.

FIG. 6 is a schematic circuit diagram showing the flow of power in the prosthetic hand system (with wrist), in which power connections to one servo motor is shown.

FIG. 7a is a photograph of the sheet metal bracket securing the battery of FIG. 5b to one of the halves of the chassis.

FIG. 7b is a photograph of PVC tubes enclosing the actuation cables for respective fingers.

FIG. 7c is a photograph showing passage of the actuating cables passing through the wrist.

FIG. 7d is a photograph of the chassis of palm with custom-designed sheet-metal brackets.

FIG. 7e is a photograph of a side view and top view of an individual sheet metal bracket.

FIG. 7f is a photograph of an air-cooled Al-alloy heat sink integrated with a microcontroller and a camera module.

FIG. 8a is a schematic that shows a controls loop outlining the sensors and actuators of the prosthetic hand system (without wrist) and flow of signals between them.

FIG. 8b is a schematic that shows a controls loop outlining the sensors and actuators of the prosthetic hand system (with wrist) and flow of signals between them.

FIG. 9a provides perspective views of three different orientations of the index finger; pins connecting phalanges are emulated by imposing concentric and fixed lateral distances between phalanges.

FIG. 9b provides plots depicting the variation of vertical distance (z) traversed by tip of finger vs. horizontal coordinate.

FIG. 10 provides front views of two different orientations of the palm during flexion-extension movement of the wrist.

FIGS. 11a and 11b provide Von-Mises Stress color charts showing finite element analysis (FEA) simulations depicting the maximum Von-Mises Stress under the application of a torsional load of 0.1 Nm, and under the application of a bending load of 0.2 kgf.

FIG. 11c provides plots depicting the variation of the minimum factors of safety (F.O.S) of respective phalanges of the pinky finger in torsion and bending vs. the applied torque and point load at tip.

FIG. 12 provides photographs showing an initial, an intermediate, and completely curled orientations of the five fingers (Index, Middle, Ring, Pinky, Thumb), indicate the individual phalanges of the pinky finger can sustain the torque until 0.25 Nm within a Factor of Safety greater than 1.5

FIG. 13 provide photographs showing an un-grasped and grasped orientations of the five fingers for selected five objects (orange, racquet ball, toothbrush, apple, and cup).

FIG. 14a provide photographs depicting the flexion, normal orientation, and back to flexion of the integrated wrist and palm.

FIG. 14b provides photographs depicting the pronation-supination movement of the assembled wrist, upon actuation by a gear motor.

FIG. 15a provides photographs used for selection of the parameter corresponding to non-maximum suppression (nms) and threshold for orange, as the distance from the camera is varied.

FIG. 15b provides photographs used for selection of the cut-off difference of the bounding box for orange.

FIG. 15c provides photographs used for selection of the parameter corresponding to nms and threshold for toothbrush.

FIG. 15d provides photographs used for selection of the cut-off difference of the bounding box for toothbrush.

FIGS. 16a and 16b are photographs depicting object recognition feedback-based grasping of an orange (FIG. 16a) and a toothbrush (FIG. 16b) by the prosthetic hand).

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure, the term “about” can allow for a degree of variability in a value or range, for example, within 10%, within 5%, or within 1% of a stated value or of a stated limit of a range.

In the present disclosure, the term “substantially” can allow for a degree of variability in a value or range, for example, within 90%, within 95%, or within 99% of a stated value or of a stated limit of a range.

A novel system and method for a prosthetic hand that can eliminate inherent latencies involved in reacting to stimuli is disclosed herein. Towards this end, a prosthetic hand system with a fast feedback is disclosed to overcome the traditional latencies of the prior art robotic arms. An integrated image recognition and feedback system (referred to as the image recognition integrated service (IRIS)) is, thus, disclosed for the prosthetic hand system which offers a low-cost end-to-end feedback driven image-sensor integrated in the palm of the prosthetic hand system which is equipped with fingers, and modular wrist, which can be integrated to the arm of a disabled human subject, e.g., a child. The IRIS system acquires successive images from an object of interest that is located in the proximity of the prosthetic hand system and uses a neural network to first recognize the object and second provide feedback signals to a controller that is configured to control the operation of the prosthetic hand system.

Two scenarios are considered herein: 1) a human subject requires a functional palm and five fingers; and 2) a human subject requires a functional wrist, palm, and five fingers. The prosthetic hand system is designed to have a total weight of less than 1 kg. The designed fingers, palm, and wrist are configured to grasp an object of interest, apply force, and lift the object. The portable hand is battery powered and is designed to operate for a predetermined amount of time under a predetermined amount of load. The prosthetic hand system has 5 functional fingers and palm to replicate standard human grasping actions. For users that require a wrist, a 4-axis modular wrist is disclosed to be integrated to the palm and five fingers. Furthermore, the designed prosthetic hand system is configured to recognize objects located less than about 30 cm. Components (e.g., sensors and actuators) are integrated inside the prosthetic hand system to ensure ease of usability, repairability, and packaging. Materials used in building an actual reduction to practice are selected to be non-flammable without any toxicity. As a result, the prosthetic hand system of the present disclosure is configured to sustain a load of about 2 kg, according to one embodiment, however, other higher loads may be achieved by proper sizing of the components. The 2 kg load is chosen, since loads typically do not exceed 2 kg (e.g., average weight of a baseball is 150 gms) in a typical usage scenario. Hence, the chosen material of the functional components is Polylactic acid, which is commonly referred to as PLA, which is a bioplastic and thermoplastic material made from natural constituents such as corn starch. PLA has a tensile strength of about 30 MPa. At a tensile strength of 30 MPa, the low density of 1.02 gm/cc ensures a light-weight design, which satisfies a critical design requirement. The components of the prosthetic hand system can be manufactured using an injection molding process or 3D printing process.

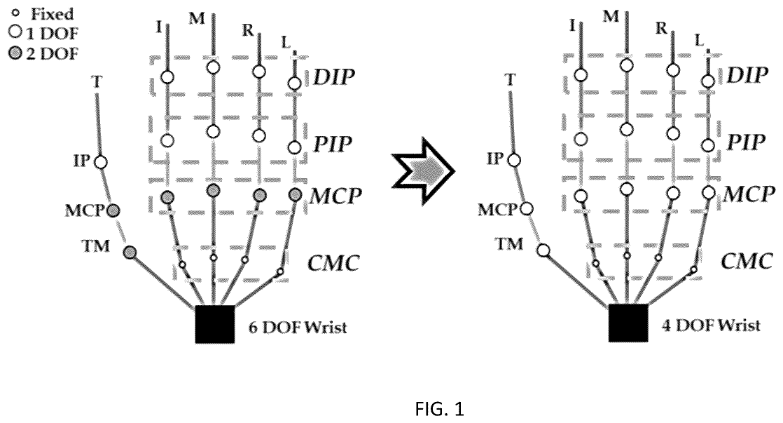

The finger joints as studied from a human hand (see FIG. 1, which provides two schematics of a human hand having 27 DOF, translated to the targeted prosthetic hand system of the present disclosure having 20 DOF) depicts that the index, middle, ring, and ring fingers each have four joints, leading up to the wrist: 1. DIP (distal-interphalangeal; 1 DOF), 2. PIP (proximal-interphalangeal; 1 DOF), 3. MCP (meta-carpophalangeal; 2 DOF), and 4. CMC (carpometacarpal; Fixed). The range of motion of the abduction-adduction of the MCP joint in adult humans is negligible; hence previous researchers have designed the MCP joint of each prosthetic finger to emulate only a single degree of freedom. The human thumb has three joints, leading up to the wrist: 1. IP (interphalangeal; 1 DOF), 2. MCP (meta-carpophalangeal; 2 DOF), 3. CMC (carpometacarpal; 2 DOF). In humans, the range of abduction and adduction motion of the CMC joint of the thumb negligibly affects certain regular actions (e.g., grasping). Moreover, in children aged between 7 and 14, it is projected that the actual range of abduction and adduction would be lesser relative to the adult humans. Hence, the CMC joint of the thumb of the presently designed prosthetic hand system has only 1 DOF. This, in turn, ensures that the thumb can be actuated using only a single motor, thus lowering the weight and subsequently rendering simplicity of the design (as is further discussed below).

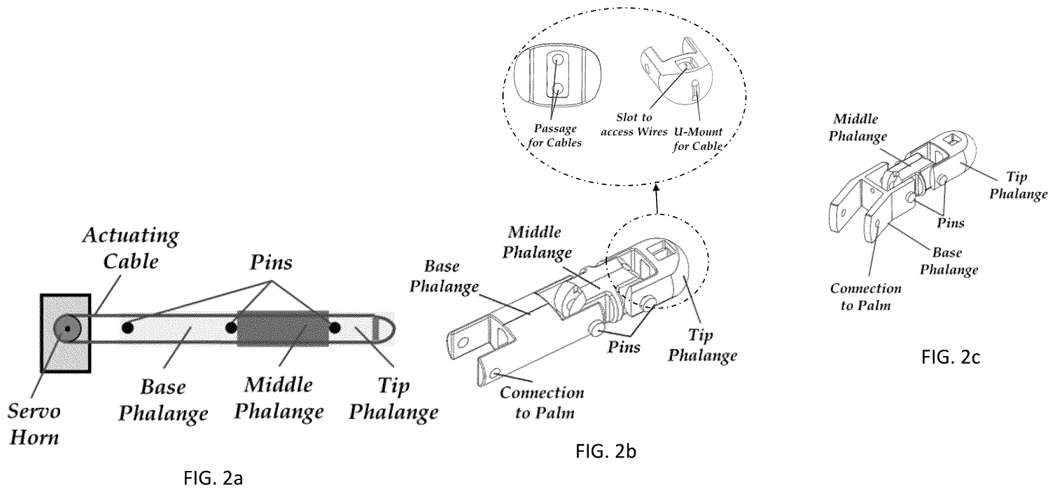

In the prosthetic hand system of the present disclosure, all four fingers (index, middle, ring, and pinky) except the thumb have similar design of the phalanges, which are only scaled differently in their respective lengths and mean diameters. A 2D planar sketch of a representative finger depicts the passage of the cable through the phalanges and the 3D view of the index finger with various features and the Thumb is shown in FIGS. 2a, 2b, and 2c (FIG. 2a is a 2D schematic of the actuation mechanism, showing the passage of the cables tied to the servo, FIG. 2b is 3D view of the index finger with various features shown in the panel, and FIG. 2c is 3D view of the Thumb with various features). Each of these four fingers comprise three phalanges (tip, middle, and base) which are connected by pins emulating a 1 degree of freedom (DOF) joint. The base phalange of each finger is coupled to the chassis of palm by a pin, thereby enabling 1 rotational DOF. For the thumb, the base phalange has a different form relative to the other phalanges. While the base phalange of the index, middle, ring, and pinky fingers has a uniform straight cross-section, the cross-section of the base phalange of the thumb has a bend half-way through towards ensuring that a grasp can be formed when the thumb has completely curled. The tip phalange of each finger has a slot that can help access the actuating cable for ease of assembly. It has two holes and a U-shaped vertical mount shown in the panel of FIG. 2b that enables the actuating cable to pass through and loop back through the passages in the middle and base phalanges. The cable passes over the U-mount in each fingertip, as depicted in FIG. 2b and is tied to both ends of the servo horn with small, high-stiffness springs to ensure that the actuating cables retain their tension. Finally, the dimensional tolerances in manufacturing individual phalanges are accounted for, by allowing clearances in the design. The tolerance in a 3D printing process was approximated and later verified by the printing process to be 0.25 mm (half the diameter of the nozzle of the 3D printer). It is important to note, that such a low tolerance is practically achievable by industry-scale injection molding processes, thereby either manufacturing processes yield similar and compatible tolerances.

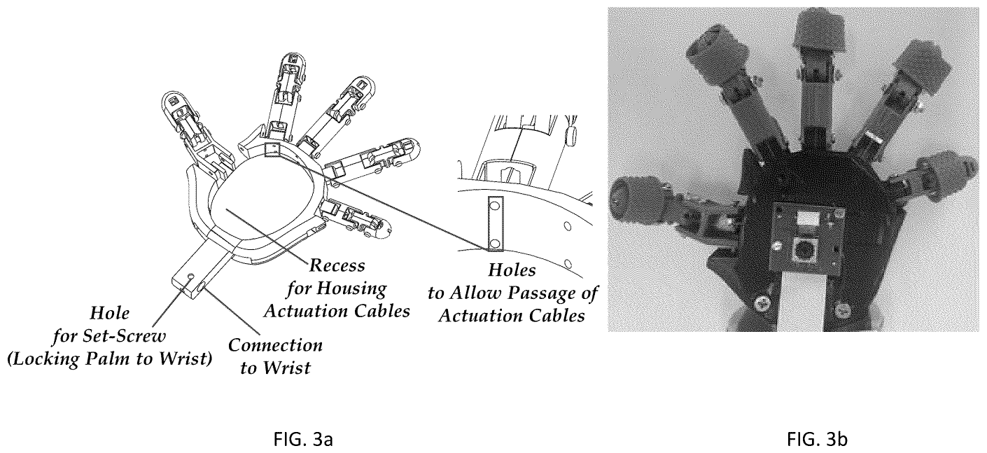

A palm design is also disclosed in FIGS. 3a and 3b (in which FIG. 3a is 3D view of the palm and integrated fingers with a magnified view of the holes in chassis to enable passage of actuation cable, and FIG. 3b is a photograph of an initially assembled prototype with the embedded camera including a palm and five fingers). Grippers (in red) are included to enhance the functionality of the fingers so as to improve gripping an object) which is configured to accommodate the robotic fingers shown in FIGS. 2a-2c. The palm houses the actuating cables for respective fingers and also reserves a space for the image sensor (camera) to be mounted on the surface of the palm. The knuckles for each finger are designed to ensure that the fingers can be securely mounted and form a closed grip, alongside enabling the passage of the actuating cables.

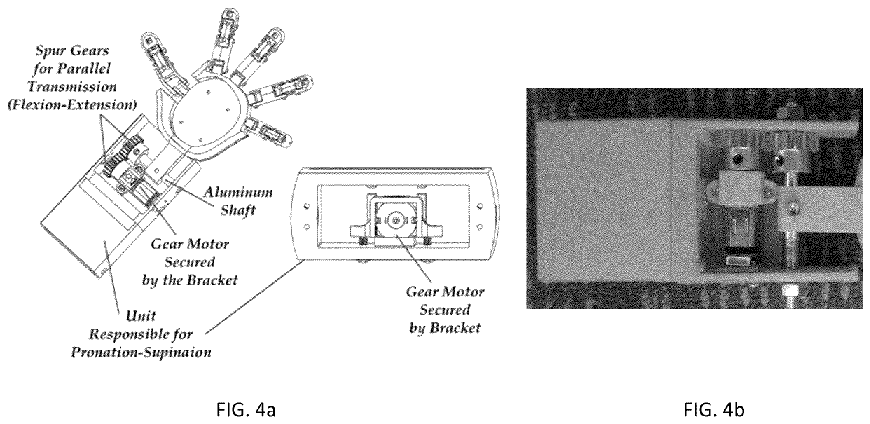

A 4 DOF wrist is also disclosed and shown in FIGS. 4a and 4b (FIG. 4a is a 3D view of the integrated wrist, palm and five fingers with various labelled features, while FIG. 4b is a photograph of an initially assembled modular wrist prototype) that is adapted to perform rotational motion about two different axes: 1. Flexion-Extension and 2. Pronation-Supination. The wrist has two distinct parts: the unit directly connecting to the palm enables the flexion-extension motion of the wrist, while the adjacent unit enables the pronation-supination movement of the wrist. The lateral dimension of the wrist is chosen to enclose the palm, while the thickness is chosen to emulate the design of a wrist of 7-14-year-old child, according to one embodiment. The palm connects with the wrist by a partially threaded aluminum shaft with counter-threaded hex nuts at the end. The set-screw locks the palm to the aluminum shaft, thereby enabling rotation only when an external torque is applied. The flexion-extension motion of the wrist is enabled using a micro-metal gear motor (discussed below), secured by a bracket and L-shaped mount to the wrist. The micro-metal gear motor is co-axially mounted with a parallel 1:1 spur-gears made of brass which can sustain torques up to 1 Nm (about 5-10 times higher than the torque generated by the gear motors). The spur gears are locked to the shafts of the gear motor and the partially threaded aluminum shaft using set-screws and can transfer rotational motion in parallelly, when the motor shaft rotates. The length of this unit is selected to allow the rotation of the palm between 30 degrees of flexion and 10 degrees of extension, and the gears are chosen to fit inside the available space, allowing the gear motor (see Section 2.3 for selection details) to be securely mounted. Next to the unit enabling extension-flexion, there is the second part of the wrist which houses a gear motor, secured using a bracket, similar to the unit that enables flexion-extension. The co-axial configuration of the both units of wrist and the axial symmetry ensures that the range of pronation-supination movement, provided by the wrist is −180 to +180 degree, once the shaft of gear motor is mounted, while maintaining the axis symmetry. Finally, slots are fabricated in the components of the wrist to ensure passage of the actuation cables and electrical wires coming from respective gear motors.

For each of the fingers, a servo motor with actuator cables is required for actuation. The chosen actuator cable is a 0.5 mm diameter monofilament fishing line (SHUR STRIKE 30 lb) which can sustain tensile loads up to 13.6 kg. The servo motor is TOWERPRO SG90 servo, as it provides a peak torque of 0.18 Nm at an operating voltage of 4.8 V and weighs 9 gm. In this design, the servo motor operates at 5 V input voltage and its maximum current draw is 250 mA. Thus, total peak power consumption of 5 servo motors is 6.25 W. One consideration is the slacking of the cables due to the fatigue induced by the various loading cycles, the prosthetic hand system can be subjected to, during the entire period of usage. However, under the applied torques, with the tensile strength of Nylon being about 70 MPa, the fishing line would likely sustain infinite life. Other servo motors (e.g., popular models include HS-5070MH, HSR-2645CR, HS-1425CR, MG996R, and RB-149416) may also be an appropriate substitute.

A micro-controller is also utilized in the disclosed prosthetic hand system which is adapted to send control signals to the different motors as well as a micro-processor to process the sensory information and feed it to the actuators or the micro-controller. A RASPBERRY PI Model 4B was chosen as a micro-controller/processor for the prosthetic hand system of the present disclosure (although other micro-controller/micro-processor may be a proper substitute). Its distinguishing features are the capability of sending control pulse width modulated (PWM) signals required for actuation and it can also interface easily with a large selection of cameras to receive the necessary sensory information to provide to the actuation feedback loop. The RASPBERRY PI operates at 5 V input voltage and has a peak current draw of 3 A. Thus, the peak power consumption of the RASPBERRY PI Model 4B is 15 W.

For the image sensor, an ARDUCAM (5 MP version) was selected (although other options may include USB cameras offering auto-focus capabilities, MICROSOFT KINECT camera which is a highly regarded choice for image-processing and object-recognition applications because it can simultaneously provide depth information alongside RGB images, as well as other cameras known to a person having ordinary skill in the art, which are suitable for the prosthetic hand system). The ARDUCAM occupies a significantly smaller volume and is lightweight relative to the former options, offers a resolution similar to web-cameras, and is the least expensive amongst all of the explored options. Also, it can easily interface with the RASPBERRY PI via I2C interface (which is a synchronous, multi-controller/target, packet switched, single-ended, serial communication protocol) with ribbon cable and function at the processing speeds of the RASPBERRY PI.

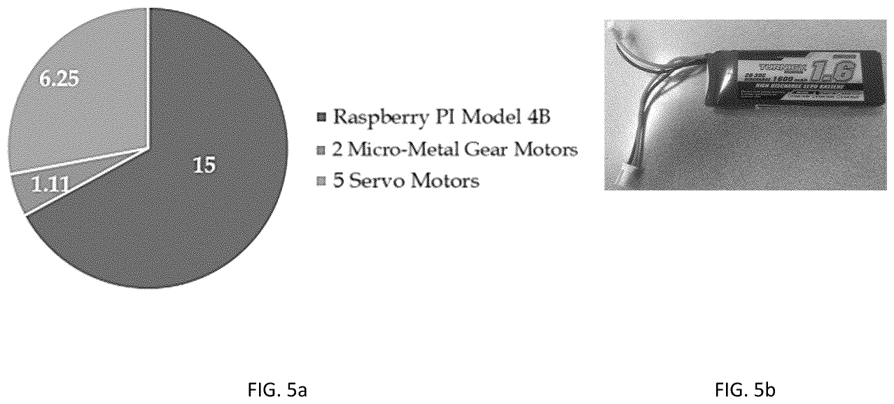

Lithium-ion polymer (LiPo) batteries were used as a power source because of high energy density and high discharge rate. The power required by the electrical and electronic hardware is depicted by a Pie-chart in FIG. 5a which is a pie-chart showing the breakdown of power in Watts, required by electrical and electronic hardware. It should be noted that the Pie-chart shown in FIG. 5a is for illustrative purposes to show how battery storage is determined and how a custom or off-the-shelf battery is chosen. Therefore, no limitation should be assigned as a result of the Pie-chart shown in FIG. 5a, to a specific implementation. A photograph of the battery is shown in FIG. 5b. As with FIG. 5a, no specific limitation should be assigned as to the type of battery needed for a specific implementation. Thus, the battery shown is for illustrative example only. The total cumulative power consumption of the hardware is 22.36 W. In the initial iteration, power source chosen to supply the total power required are 2 LiPo batteries (TURNIGY 20-30 1600 mAH) connected in series, with a mechanical ON-OFF switch in between. Each of these batteries has 2 cells and provides a peak voltage of 7.4 V. The cumulative W-hr rating of the 2 batteries (4 cells) is 23.68 Wh, such that the entire prosthetic hand could operate for 1 hr and 4 min at continuous peak power throughput.

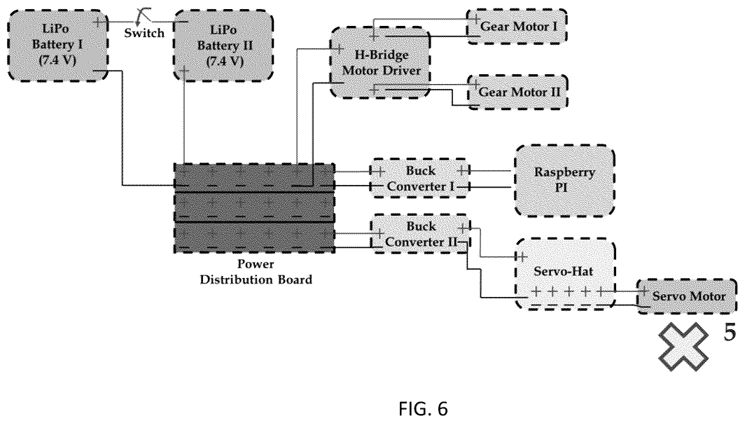

To accommodate different operating voltages required by the different components, a power distribution board is needed to convert the input power from the battery to provide the electrical power to the respective components using DC-DC converter. It is important to note that the non-isolated DC-DC converter requires capacitors and current-limiting resistors along with a printed circuit board (PCB) to provide the step-down functionality. The linear regulator requires capacitors and current-limiting resistors, an externally mounted heat sink, along with a PCB to provide the step-down functionality. For the power-electronic circuitry, two LiPo batteries are connected in series through a mechanical switch, thereby generating a total maximum average voltage of 14.8 V, which powers an H-Bridge. Separate buck-converters are used to step down the maximum input voltage of 14.8 V to 5 V for powering the RASPBERRY PI and the Servo-hat respectively. All connections are made through the power distribution board. The circuit diagram interconnecting these various components, is depicted in FIG. 6 which is a schematic circuit diagram showing the flow of power in the prosthetic hand system (with wrist), in which power connections to one servo motor is shown; and remaining 4 are coupled in similar fashion). Example of non-isolated DC-DC converter is TRACO POWER TSR 2-2425, and DC-DC module is LY-KREE K120508,

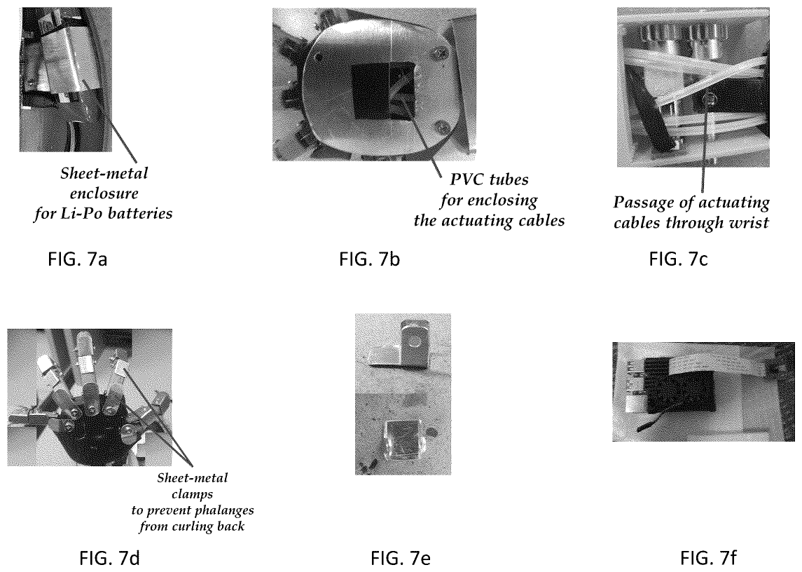

The electrical and electronic hardware discussed above are housed inside a chassis made of PLA, according to one embodiment. The chassis has two halves. The first half houses the servo motors, power distribution board, H-Bridge, and one of the batteries. The second half houses the other battery, the RASPBERRY PI unit, and the servo-hat. Space is allocated in the chassis for the passage of electrical wires connecting to the electronic hardware. Metal brackets are used each has the matching holes for the pins to pass through and ensure that consecutive phalanges remain connected and perform the curling motion. The lateral spacing between the servos for actuating respective fingers is decided to ensure that the actuating cables have space to allow the servo horns rotate around their required range of motion. Also, for actuating cables running to each finger, the cables are passed through cylindrical plastic Polyvinyl chloride (PVC) tubes of 1 mm internal diameter to prevent entanglement as the components of the wrist rotate. These tubes pass through the chassis of the palm and the wrist to the respective servo motors. For the 3D-printed fingers, the phalanges by themselves do not have any capability to restrict them from curling backwards beyond their level position relative to the palm. Hence, aluminum sheet metal brackets are custom-designed and integrated at three different locations along each finger. Also, to accommodate for slight expansion of batteries from internal heating lightweight (0.5 mm-thick) aluminum sheet metal (density: 2.7 gm/cc, ultimate tensile strength: 310 MPa) were used for securing the batteries to the inside of the chassis. When the Raspberry PI runs image-recognition and distance—measurement algorithms, the maximum temperature of the processors would reach the about 80° C. threshold. Hence, air-cooled heat sinks were utilized to prevent throttling of performance. Air-cooled Al-alloy heat sinks (GEEKWORM) are integrated to the RASPBERRY PI, followed by securing them to the chassis using a combination of Aluminum sheet, screws and nuts. The total measured weight of the actually reduced to practice prosthetic hand system is about 750 gms. Reference is made to FIGS. 7a, 7b, 7c, 7d, 7e, and 7f which depict the components described above. Specifically, FIG. 7a is a photograph of the sheet metal bracket securing LiPo battery to the one of the halves of the chassis, FIG. 7b is a photograph of the PVC tubes enclosing the actuation cables for respective fingers, FIG. 7c is a photograph showing passage of the actuating cables passing through the wrist, FIG. 7d is a photograph of the chassis of palm with custom-designed sheet-metal brackets, FIG. 7e is a photograph of a side view and top view of an individual sheet metal bracket, and FIG. 7f is a photograph of the air-cooled Al-alloy heat sink integrated with the RASPBERRY PI and the camera module.

In the prosthetic hand system, each finger is controlled by the actuating fishing line that is tied at both ends of the servo horn. The angular position of the servo horn decides the orientation of each finger. To control the angular position of the servo horn, pulse width modulated (PWM) signals are sent from the 16 PWM Channel Servo-hat (manufactured by ADAFRUIT), which interfaces with the RASPBERRY PI via I2C communication protocol. For the wrist, which enables rotation about two axes, separate gear motors are required to modulate rotations about separate axes. The two gear motors are wired to an L298 Dual H Bridge, manufactured by QUNQI. For each motor, the L298 Dual H bridge receives two digital and one analog input (PWM Signal) from the RASPBERRY PI. The relative polarity of the digital inputs decides the direction of rotation of the gear motor and the amplitude of the PWM signal dictates the speed of rotation of the gear motor and the respective section of the wrist.

The prosthetic hand system receives sensory input from the camera mounted on the palm of the prosthetic hand. The camera provides two specific inputs: 1) the type of object (e.g., balls of different types, a pen, a cup, a glass, tools of different types, etc.), and 2) the distance from the lens of the camera (located at the palm) to the object. The object is detected via a neural network, e.g., SSD MOBILENET-V3. This model is trained on 91 total categories of objects in one of the most exhaustive and widely used data sets which comprises the common objects in context.

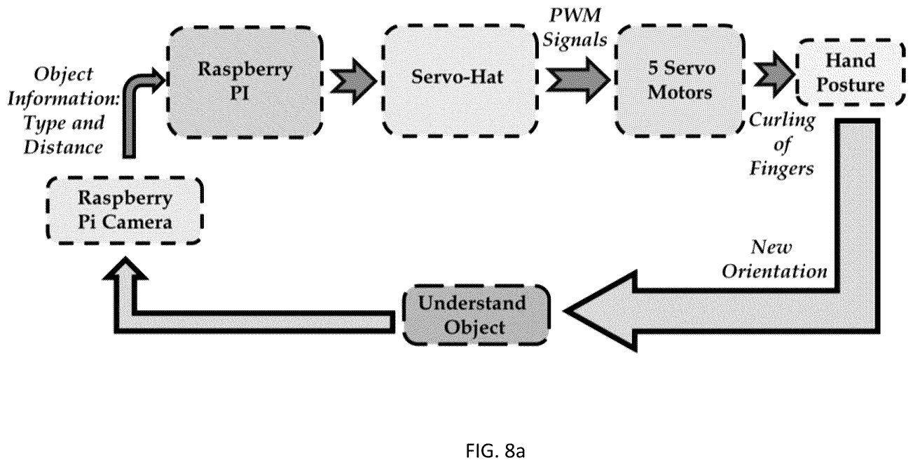

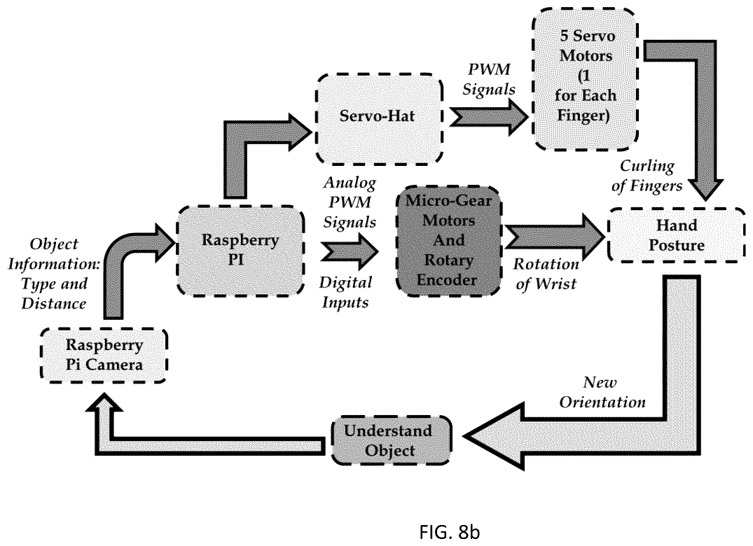

There are two critical input parameters for the neural network: 1. threshold, which corresponds to the probability that the neural network has been able to classify that object, and 2. non-maximum suppression (nms) which determines the ability to detect a single instance of object amongst multiple overlapping objects. For a given object, once this threshold has been set, the neural network is trained to recognize all objects within that threshold. In the present disclosure, the distance measurement has been integrated with this object-recognition algorithm. For purposes of measuring the distance of an object from the image sensor, the coordinates of the bottom left and top-right of a given bounding box are employed to measure the difference in width coordinates of the bounding box in pixels. As an object approaches closer to the prosthetic hand system, the bounding box becomes larger and thus the difference increases, until it occludes the field of view of the camera. For every object, a cut-off difference of the bounding box is obtained from testing with the camera sensor, followed by invoking the occlusion criterion to ensure the corresponding grasp. In the software algorithm a metric is used to store the analog value of the difference of the bounding box and becomes 0 when the frame of camera is occluded by the object. The integration of the sensor and the actuator loops is depicted in FIGS. 8a and 8b. Specifically, FIG. 8a is a schematic that shows a controls loop outlining the sensors and actuators of the prosthetic hand system (without wrist) and flow of signals between them, while FIG. 8b is a schematic that shows a controls loop outlining the sensors and actuators of the prosthetic hand system (with wrist) and flow of signals between them.

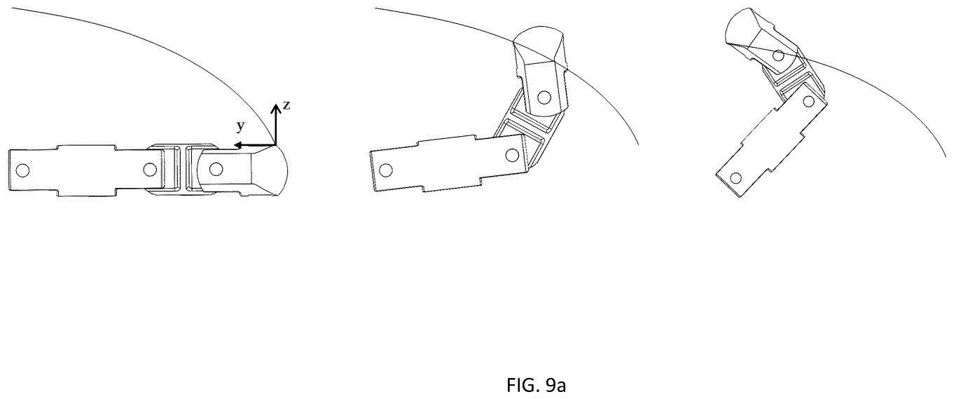

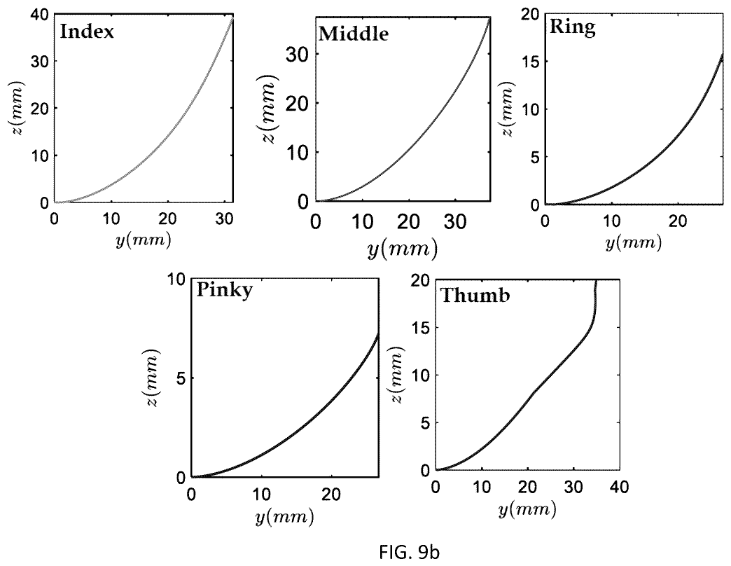

Mechanical interference between phalanges in the fingers is of concern and is prevented by studying the vertical motion of the fingertip as it travels horizontally. If the tip of the finger can successfully curl up, this means that there is no mechanical interference between phalanges. Referring to FIG. 9a, perspective views of three different orientations of the index finger are shown; pins connecting phalanges are emulated by imposing concentric and fixed lateral distances between phalanges. Black line denotes the traced path by the tip of the finger. Referring to FIG. 9b, plots depicting the variation of vertical distance (z) traversed by tip of finger vs. horizontal coordinate are provided.

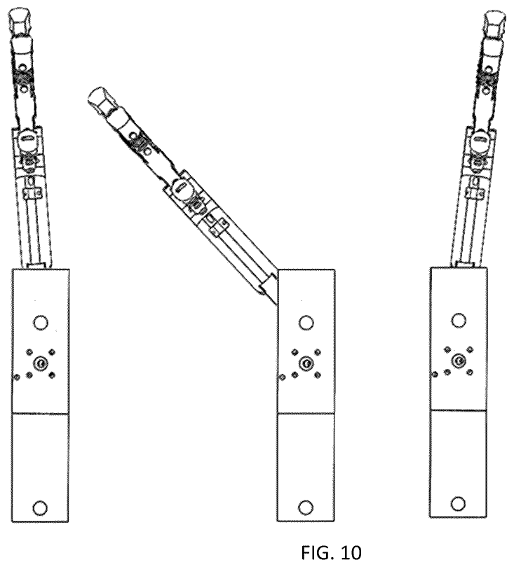

Mechanical interference between connected parts of the wrist is also of concern which is prevented by studying the degree of rotation of a given part vs. the rotation of the shaft of the gear motor responsible for driving that part. After iterations in design, the trajectory of the two separate components of the wrist responsible for flexion-extension and pronation-supination along with the different orientations of the parts is depicted in FIG. 10 which offers front views of two different orientations of the palm during flexion-extension movement of wrist. Available theoretical range of rotation during flexion-extension is about 30 degrees of flexion to about 10 degrees of extension. The pronation-supination movement of the wrist is trivially satisfied by the design as the two units of the wrist have the same average height and axially symmetric, providing a theoretical range of rotation during pronation-supination to be −180 to 180 degrees, which is more than the range of pronation-supination of an actual human wrist.

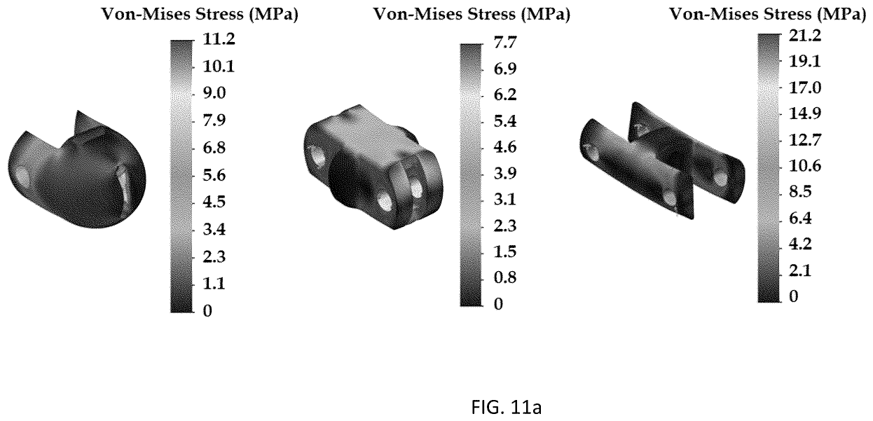

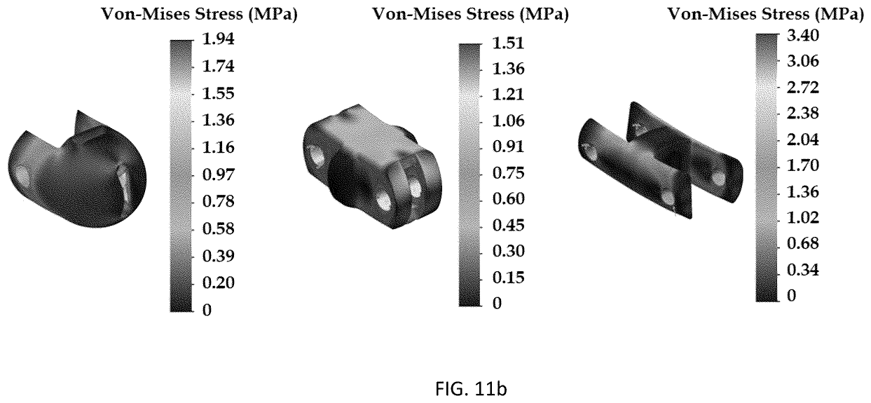

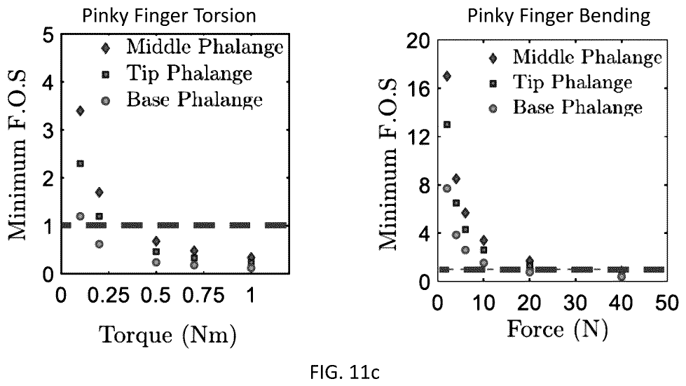

Referring to FIGS. 11a, 11b, and 11c, simulation stress results are provided. Specifically, FIGS. 11a and 11b provide Von-Mises Stress color charts showing FEA simulation depicting the maximum Von-Mises Stress under the application of a torsional load of 0.1 Nm, and under the application of a bending load of 0.2 kgf. FIG. 11c provides plots depicting the variation of the minimum factors of safety (F.O.S) of respective phalanges of the pinky finger in torsion and bending vs. the applied torque and point load at tip. The grey line in both the plots correspond to a factor of safety of 1.

The curling of individual fingers of the prosthetic hand system of the present disclosure relies on the functionality of the phalanges constituting each finger. During the usage of the prosthetic hand system, the fingers might be subjected to both external forces and internal impacts. Amongst all the fingers, the pinky finger has the least average diameter and thickness and hence, is most prone to failure. Two different cases are considered: 1) when there is a torque acting at the tip of each phalange, while the other end (pin joint) is fixed, and 2) when a load acts at the tip of each phalange, while the other end (pin joint) is fixed. Case 1 can arise, when the mechanism gets locked due to errors in tolerances. Case 2 can potentially arise in case of point loads/impacts at the tip of each phalange. Both these cases are conservative, as the other end (pin joint) is considered to be fixed.

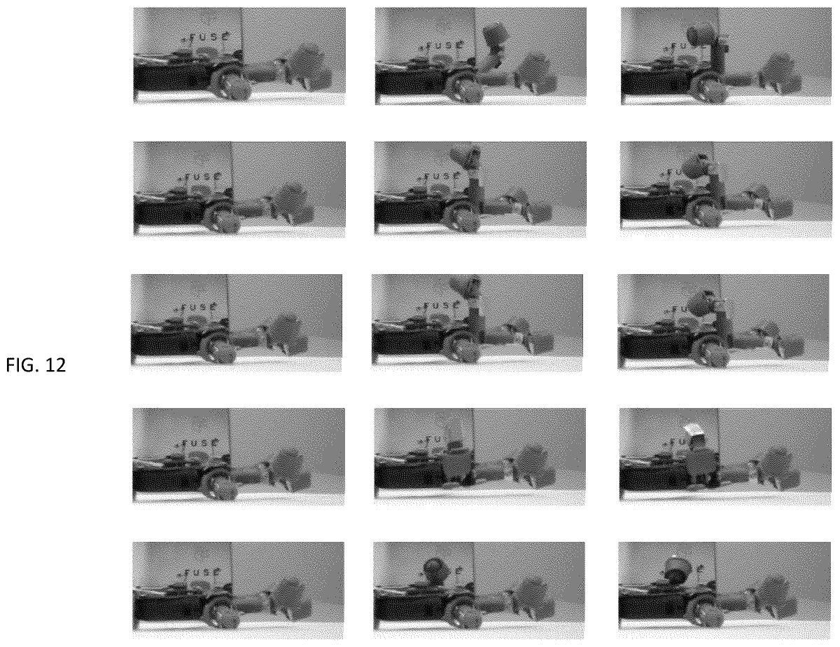

Results related to case 1 are shown in FIG. 12 which are photographs showing the initial, an intermediate, and completely curled orientations of the five fingers (Index, Middle, Ring, Pinky, Thumb), indicate the individual phalanges of the pinky finger can sustain the torque until 0.25 Nm (>torque generated by the TOWERPRO SG90 servo), within a Factor of Safety greater than 1.5. This, in turn, ensures that the phalanges of all the other fingers can definitely sustain a torque till 0.25 Nm, within higher Factors of Safety, because of higher average diameter. Results related to case 2 (see FIG. 12) indicate the individual phalanges of the pinky finger can sustain the force until 2 kgf (about 10× weight of a baseball that is 150 gms), within a Factor of Safety >3. This, in turn, ensures that the phalanges of all the other fingers can definitely sustain a force till 2 kgf, within higher Factors of Safety, because of higher average thickness.

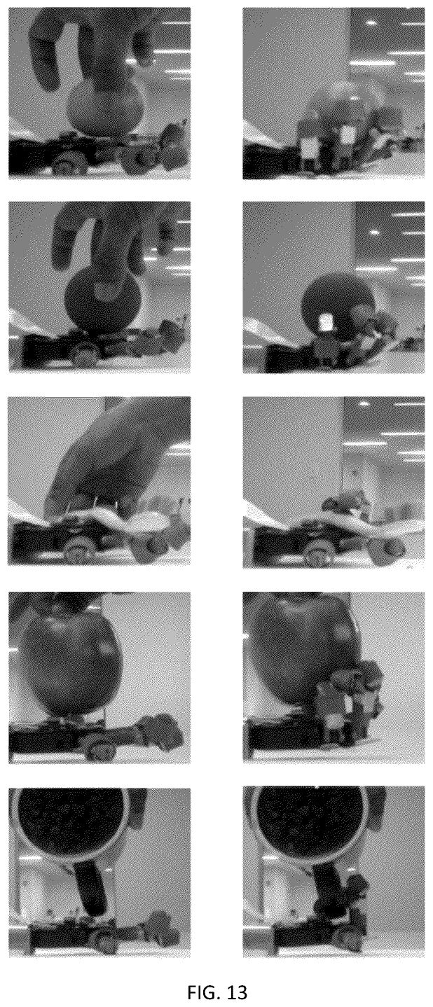

To ensure the fingers of the prosthetic hand system can successfully grasp various objects of interest (an orange, apple, a cup, a toothbrush, and a racquet ball), the actually reduced to practice prosthetic hand system was tested. Referring to FIG. 13, photographs are provided showing the un-grasped and grasped orientations of the five fingers for the selected five objects (orange, racquet ball, toothbrush, apple, and cup). Un-grasped configurations are represented by images on the one side of FIG. 13 and grasped configurations are represented by the images on the other side of FIG. 13. The apple and cup are not per the size for the prosthetic hand system for a child, hence are supported with a human hand.

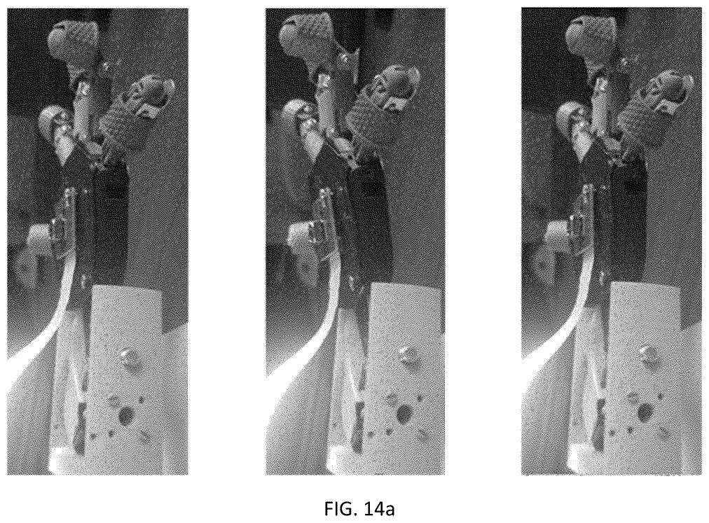

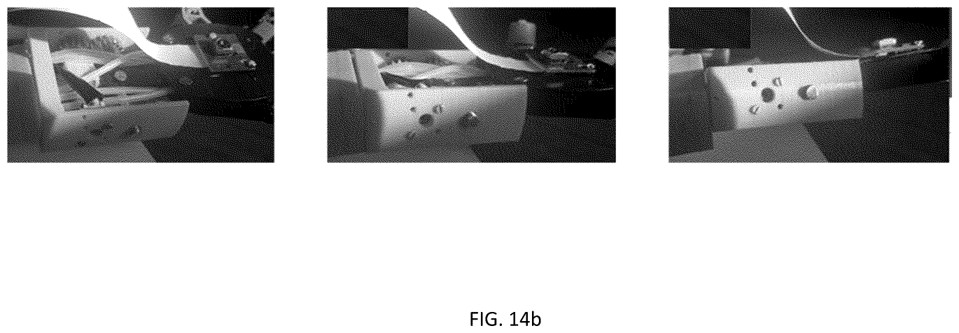

With the grasping test completed, the different motions (flexion and pronation-supination) of the wrist with integrated palm and fingers was tested, results of which are provided in FIGS. 14a and 14b (which FIG. 14a are photographs depicting the flexion, normal orientation, and back to flexion of the integrated wrist and palm; and FIG. 14b are photographs depicting the pronation-supination movement of the assembled wrist, upon actuation by a gear motor). Results depict that the wrist can perform flexion motion and this modular unit can be directly integrated to the palm and rest of the fingers. The range of extension is minimal in this assembly, because of external integrated hardware in the wrist section (e.g., tubes passing through), however, that wouldn't inhibit grasping of objects. The range of pronation-supination available is lower relative to the theoretical range, because the passage of tubes inhibits the complete rotation. Also, the tests suggest that because of the external load attached to the chosen gear motors, a 4 cell LiPo battery with a higher discharge may be preferred relative to 2 LiPo batteries in series.

As discussed above, the 5 MP camera sensor accomplishes two different tasks: 1. object recognition, and 2: distance measurement. Although the camera sensor comes with the autofocus capability and can handle frame sizes till 1280×1280, a 320×320 frame size is used without any external algorithm for autofocus. This is because the framerate reduces by 67% as the frame size changes from 320×320 to 1280×1280, which would introduce latencies in the sensor-actuator loop of the prosthetic hand system. Similar reduction in the frame-rate is observed if attempting to control the focus of the camera for detecting the object. The object recognition is accomplished by the MOBILE NET SSD-v3 algorithm which has two specific inputs (threshold and non-maximum suppression). These two inputs decide the performance of the algorithm, for a specific hardware and the external conditions (e.g., illumination). The objective of this testing is to ensure that there are no overlapping bounding boxes as an object approaches the camera. Overlapping bounding boxes would provide unreliable sensor inputs to the actuators, resulting in un-controlled behavior of the prosthetic hand. Testing is performed for two objects: 1. orange and 2. toothbrush. Tests at different distances of the camera from the object, reveal that for orange, under the normal illumination conditions, prevalent in a community center, the chosen value of the threshold and the non-maximum suppression are 0.4 and 0.2, respectively. The corresponding selected values for the toothbrush are 0.5 and 0.1, respectively. At these optimum values, the cut-off difference of the bounding box for an orange is 285 and for the toothbrush is 293, for the chosen frame length of 320.

In addition to the camera sensor, other sensors such as force sensors, may be deployed. The sensors are deployed in the feedback loop for control of the actuators as well as according to one embodiment for modification of the neural network parameters.

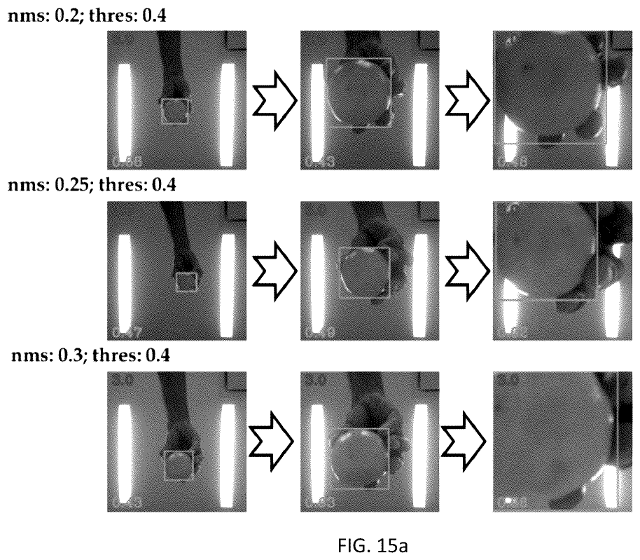

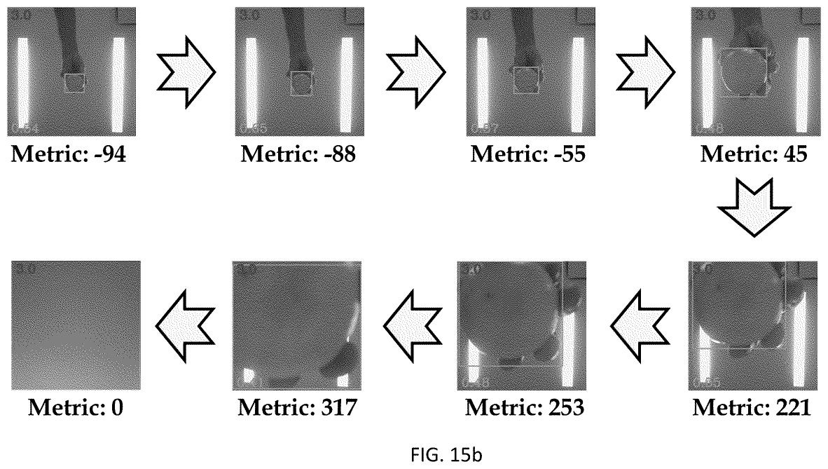

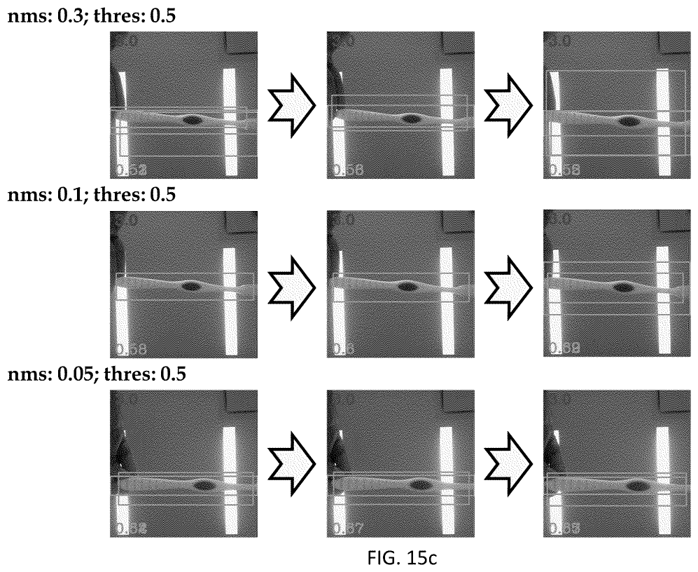

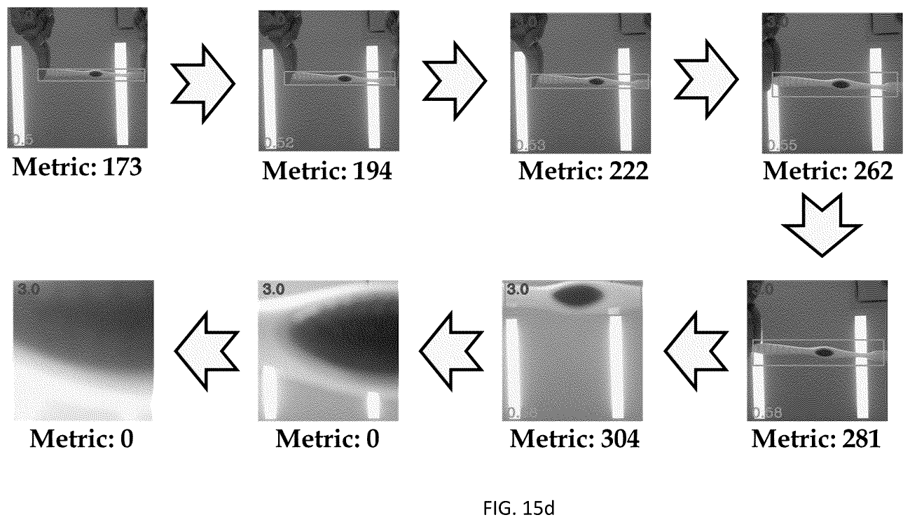

Referring to FIGS. 15a-15d, the results of the test are provided. Specifically, FIG. 15a provides photographs used for selection of the parameter corresponding to nms and threshold for orange, as the distance from the camera is varied; FIG. 15b provides photographs used for selection of the cut-off difference of the bounding box for orange; FIG. 15c provides photographs used for selection of the parameter corresponding to nms and threshold for toothbrush; and FIG. 15d provides photographs used for selection of the cut-off difference of the bounding box for toothbrush. All images are extracted from videos, which are recorded at the framerate, specified in the images.

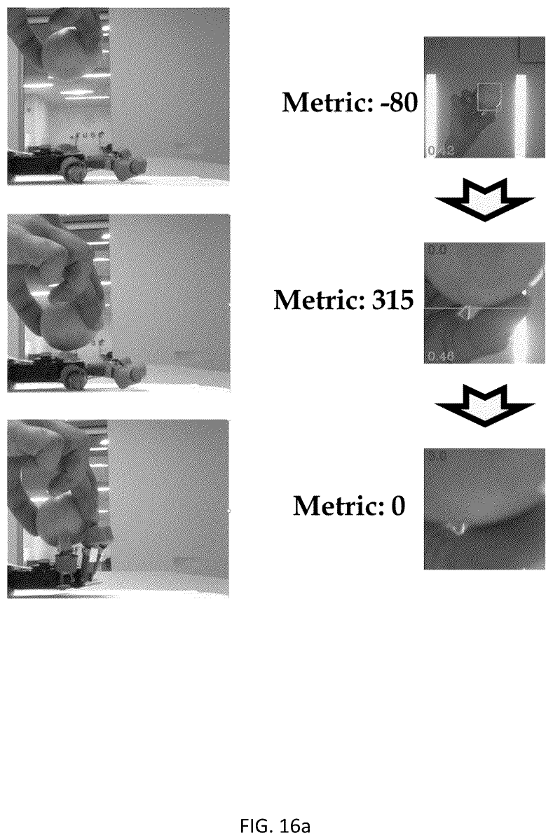

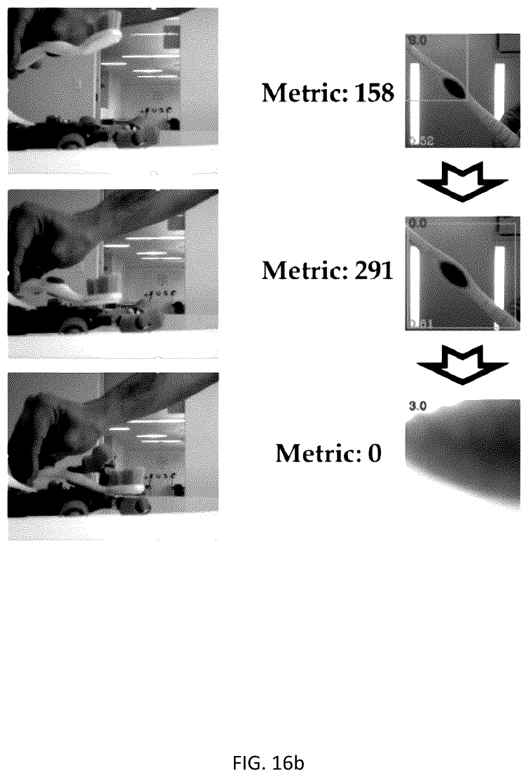

The object recognition feedback-based actuation of the prototype is demonstrated with two objects: 1. orange and 2. toothbrush, towards demonstrating the two different modes of grasp, namely, power and precision. Results depict that the prosthetic hand can intercept feedback from the camera and can grasp objects of different form-factors (complete grasp, as seen in FIGS. 16a and 16b which are photographs depicting object recognition feedback-based grasping of an orange (FIG. 16a) and a toothbrush (FIG. 16b) by the prosthetic hand).

While a prosthetic hand system is disclosed in the system, the disclosed system may also be configured in the shape of a glove that is wearable by a human subject. The same basic structure shown can be implemented but instead of solid fingers, palm, and wrist, the assembly may be hollow and thus wearable by a human subject who has lost control of his/her hand. This embodiment may be specially advantageous for stroke patients both in therapy as well as everyday enhancement of life.

It should be noted that while a number of commercially available products have been called out in the present disclosure, no limitation is intended as to use of such commercially available products. In all cases, other custom or commercially available products may be substituted with similar or even better performance.

Those having ordinary skill in the art will recognize that numerous modifications can be made to the specific implementations described above. The implementations should not be limited to the particular limitations described. Other implementations may be possible.

1. A prosthetic hand assembly, comprising:
a hand structure including an actuatable wrist, a palm, and a plurality of actuatable fingers, wherein the actuatable wrist and at least one of the plurality of actuatable fingers is coupled to corresponding actuators configured to selectively direct movement of the actuatable wrist and at least one of the plurality of actuatable fingers;
a camera disposed on one side of the hand structure, wherein the camera is selectively operable to generate a plurality of images of an object positioned adjacent the hand structure;
a processor coupled with the actuators, wherein the processor is communicatively coupled with the camera and configured to receive the plurality of images of the object, wherein the processor is configured to determine one or more characteristic of the object from the plurality of images of the object, and wherein the processor is configured to selectively drive one or more of the actuators to affect movement of the actuatable wrist and at least one of the plurality of actuatable fingers based on the one or more characteristics of the object.

2. The prosthetic hand assembly of claim 1, wherein the one or more characteristics of the object includes a distance from the camera to the object.

3. The prosthetic hand assembly of claim 2, wherein the processor is configured to generate a bounding box for each image of the plurality of images to thereby create a plurality of bounding boxes, wherein each bounding box is based upon at least two coordinates of the object in each image of the plurality of images, wherein the distance of the palm of the hand structure from the object is determined based upon a comparison of each bounding box of the plurality of bounding boxes.

4. The prosthetic hand assembly of claim 1, wherein the one or more characteristics of the object includes type of the object.

5. The prosthetic hand assembly of claim 4, wherein the type of object includes balls of different types, a pen, a cup, a glass, and tools of different types.

6. The prosthetic hand assembly of claim 4, wherein the actuators are activated based on the type of the object.

7. The prosthetic hand assembly of claim 6, wherein force of the actuators is adjustable based on the type of the object.

8. The prosthetic hand assembly of claim 1, further comprising sensors with signals therefrom which when integrated with the processor and the actuators provide a feedback control loop for operating the actuators.

9. The prosthetic hand assembly of claim 8, wherein the processor includes a memory, wherein the memory is configured to store a pre-trained neural network model therein, and wherein the pre-trained neural network model is configured to determine the type of the object.

10. The prosthetic hand assembly of claim 9, wherein parameters of the pre-trained neural network are adjustable based on sensor signals.

11. A method of operating a prosthetic hand assembly, comprising:
providing a hand structure including an actuatable wrist, a palm, and a plurality of actuatable fingers, wherein the actuatable wrist and at least one of the plurality of actuatable fingers is coupled to corresponding actuators configured to selectively direct movement of the actuatable wrist and at least one of the plurality of actuatable fingers;
providing a plurality of images of an object adjacent a camera disposed on one side of the hand structure, positioned adjacent the hand structure;
communicating the plurality of images to a processor coupled to the actuators, wherein the processor is configured to receive the plurality of images of the object, wherein the processor is configured to determine one or more characteristic of the object from the plurality of images of the object, and wherein the processor is configured to selectively drive one or more of the actuators to affect movement of the actuatable wrist and at least one of the plurality of actuatable fingers based on the one or more characteristics of the object.

12. The method of claim 11, wherein the one or more characteristics of the object includes a distance from the camera to the object.

13. The method of claim 12, wherein the processor is configured to generate a bounding box for each image of the plurality of images to thereby create a plurality of bounding boxes, wherein each bounding box is based upon at least two coordinates of the object in each image of the plurality of images, wherein the distance of the palm of the hand structure from the object is determined based upon a comparison of each bounding box of the plurality of bounding boxes.

14. The method of claim 11, wherein the one or more characteristics of the object includes type of the object.

15. The method of claim 14, wherein the type of object includes balls of different types, a pen, a cup, a glass, and tools of different types.

16. The method of claim 14, wherein the actuators are activated based on the type of the object.

17. The method of claim 16, wherein force of the actuators is adjustable based on the type of the object.

18. The method of claim 11, further comprising sensors with signals therefrom which when integrated with the processor and the actuators provide a feedback control loop for operating the actuators.

19. The method of claim 18, wherein the processor includes a memory, wherein the memory is configured to store a pre-trained neural network model therein, and wherein the pre-trained neural network model is configured to determine the type of the object.

20. The method of claim 19, wherein parameters of the pre-trained neural network are adjustable based on sensor signals.
